(19) United States
(12) Patent Application Publication
(10) Pub. No.: US 2011/0245878 A1
(43) Pub. Date: Oct. 6, 2011

(54) RETENTION MECHANISMS FOR ROD LOCKING SET SCREWS

(75) Inventors: Richard W. Franks, Memphis, TN (US); Jeff R. Justis, Germantown, TN (US); Jason M. May, Cordova, TN (US); Alex G. Zolotov, Collierville, TN (US)

(73) Assignee: WARSAW ORTHOPEDIC, INC., Warsaw, IN (US)

(21) Appl. No.: 12/754,289

(22) Filed: Apr. 5, 2010

(51) Int. Cl. A61B 17/70

(52) U.S. Cl. 606/278

(57) ABSTRACT

The present invention concerns spinal fixation systems, and particularly systems utilizing pedicle screws, connectors and elongated members positioned adjacent to the spinal column. More specifically, the invention concerns improvements to spinal connectors having set screws used to connect to the elongate members, such as spinal rods, that include a means for inhibiting rotational movement of a partially inserted set screw before such connectors are implanted by the surgeon.

### BACKGROUND

The present invention concerns spinal fixation systems, and particularly systems utilizing a threaded screw/bolt for the fixation of elongated rods adjacent to the spinal column and transverse connectors for connection between such spinal rods for increasing the torsional stability of the spinal stabilization system. More specifically, the invention concerns improvements to such spinal constructs such as to a transverse connector that is used to interconnect two approximately parallel elongate members, such as spinal rods, that include a means for inhibiting movement of the set screws before use by the surgeon. Such improvements may also be utilized with other spinal connectors to connect the spinal rods to a threaded screw and/or bolt and utilizing a set screw to affix the spinal rod to the spinal connector.

Spinal fixation systems are implanted during a surgical procedure to treat a variety of problems. These treatments include correction of congenital spinal deformations, repair of spinal injuries and fusion of vertebra to stabilize degenerative conditions and alleviate chronic back pain. Several techniques and systems have been developed for correcting and stabilizing the spine and facilitating spinal fusion. In one common system, a longitudinal member, such as a bendable rod, is disposed along the vertebral column and is fixed to various vertebrae along the length of the column by way of a number of fixation elements. Usually, the surgeon first attaches vertebral fixation elements to the spine in appropriate anatomic positions, and then attaches each vertebral fixation element to the spinal rod. Often times a set screw is utilized to affix the rod to fixation element.

In order to increase the torsional stability of the spinal fixation system, one or more transverse connectors may be connected across to each of the rods along the axial plane of the spine. Transverse connectors consist of one or more arms that can be locked on a rod by a set screw. The arms can be adjusted in length and are typically joined by an eyebolt component with a lock nut. However, when the transverse connectors are packaged at the manufacturing plant the set screws are preferably preassembled to the transverse connector to reduce surgery time. The sets screws are typically installed in the fully engaged position from the manufacturer to prevent the set screw from disengaging from the transverse connector during the packaging process and shipment by the manufacturer to the customer. The customer is typically a hospital which would then make the spinal components available to a surgeon for use in a spinal surgery. The hospital may additionally have all of the spinal components sterilized at the hospital in preparation for use in a spinal surgical operation. Many people are handling these spinal components before they reach the operating room and if a set screw became separated from the transverse connector the product would be unusable by the surgeon. Since the set screws are installed in the fully tightened and/or engaged position, the rod is prevented from entering into the transverse connector and therefore, the set screw must be at least partially unthreaded from the transverse connector prior to use by the surgeon. This unthreading step by either the surgeon or the hospital nursing staff may also cause the set screw to become disengaged from the transverse connector. When a hurried attempt in the operating room to reengage the set screw occurs it is possible to cross-thread the set screw which would result in a damaged transverse connector. The whole process of getting the transverse connectors ready for use in the operating room causes surgeon frustration and possibly damaged implants and additional cost to the customers and manufacturers. Current transverse connector assemblies do not provide a means to retain the set screw within the transverse connector in a manner that would allow the transverse connector to be placed over the rod without first unloosening the set screw.

### SUMMARY

According to one aspect a transverse connector for connection to a pair of spinal rods is disclosed having set screws that are engaged within the transverse connector in an open position not blocking the rod receiving channels. The set screws shown are break off set screws including a threaded portion with a depending rod engaging end and a break off head portion having a driving portion configured to receive a screw driver or the like for rotating the set screws into contact with a spinal rod. However, the invention may also encompass non-break off set screws. The various embodiments discussed below will work with such non-break off set screws as well. The transverse connector can be provided with a single span or be as shown herein having a first arm and a second arm having an eye. An eyebolt is included having a horizontal passageway for receiving at least a portion of the first arm and an upper portion for receiving the eye of the second arm. The transverse connector includes a means for inhibiting rotational movement of the set screws prior to the transverse connector being fixedly secured to the spinal rods.

In one form, the means for inhibiting comprises an oversized skirt being provided about the circumference of the set screw. The skirt will be deformed during the partial pre-installation of the set screw into the transverse connector at the manufacturer's location. The skirt can be machined into the set screw and, therefore, be made of the same material as the set screw which would typically be titanium or stainless steel. In another form, the skirt can be inserted within a groove about the circumference of the set screw. In this form, the skirt could be made from an elastic material such as a nitinol metal ring or could be an elastic polymeric material both of which would deform sufficiently to allow the set screw to be initially partially threaded into the transverse connector and to hold such set screw in that partially threaded position, but would not unduly prohibit being further completely threaded into the transverse connector by the surgeon to connect the transverse connector to a spinal rod during a spinal operation. In one representative form, the skirt is a segmented skirt which may provide for an easier deformation of the skirt and reduce the necessary torque required by the surgeon to overcome the additional elastic deformation of the skirt to complete the threading of the set screw into the transverse connector.

In another form, the means for inhibiting comprises the placement of an elastically deformable material within the thread form of the set screw. The deformable material within the thread would create a drag when initially threading the set screw a partial way into the transverse connector. The elasticity of the deformable material would hold the set screw within the threaded bore of the transverse connector. However, with additional torque of the set screw by the surgeon the set screw can be fully threaded into the transverse connector to attach same to the spinal rod. The deformable material may be an elastic polymer placed within the thread form by the manufacturer and when the set screw is initially partially threaded into the transverse connector the elastic material would create a drag on continued insertion of the set screw within the transverse connector. The surgeon could overcome this drag by utilizing additional force on threading the set screw into the transverse connector to attach same to a spinal rod.

In another form, the means for inhibiting comprises the placement of an elastically deformable peg into the transverse connector to capture a portion set screw thread when in is partially threaded into the transverse connector. The set screw would be initially threaded to a position in the transverse connector where the depending rod engaging end of the set screw is not within the channel of the transverse connector for receiving a spinal rod and then the deformable peg would inserted within the transverse connector to partially capture a thread of the set screw. The deformable peg would deform to allow the set screw to be completely installed within the transverse connector by the surgeon with additional rotational torque applied on the set screw by use of an appropriate driver to install the set screw.

In yet another form, the means for inhibiting comprises a snap ring being placed with the internally threaded hole of the transverse connector. The set screw has a circumferential groove adjacent the external thread form such that the set screw can be initially threaded into the transverse connector until the snap ring fits into the circumferential groove to hold the set screw in place partially threaded into the transverse connector. With additional torque applied to the set screw by the surgeon the set screw could be fully threaded into the transverse connector to capture the spinal rod during a spinal surgery.

And, in yet another form, a spinal connector for connection between a threaded bolt/screw and the elongated rod is disclosed having a set screw that is engaged within the spinal connector in an open position not blocking the rod receiving channel of the spinal connector. The set screw shown is a break off set screw including a threaded portion with a depending rod engaging end and a break off head portion having a driving portion configured to receive a screw driver or the like for rotating the set screws into contact with the spinal rod. The spinal connector includes a means for inhibiting rotational movement of the set screw prior to the spinal connector being fixedly secured to the spinal rods.

Related features, aspects, embodiments, objects and advantages of the present invention will be apparent from the following description.

### BRIEF DESCRIPTION OF THE DRAWINGS

In the accompanying drawings, which are incorporated in and constitute a part of the specification, embodiments of the present invention are illustrated which, together with the general description of the invention given above, and the detailed description given below, serve to exemplify the embodiments of this invention. The components in the Figures are not necessarily to scale, emphasis instead being placed upon illustrating the principles of the invention and, in the Figures, like reference numerals designate corresponding parts throughout the different views.

FIG. 1 is a top elevational view of a spinal fixation construct in accordance with one embodiment of the present invention.

FIG. 2A is a side view of a prior art transverse connector having the set screws threaded completely therein of the type that could be used in a spinal construct as shown in FIG. 1.

FIG. 2B is a side view of a prior art transverse connector having the set screws only partially threaded into the transverse connector of the type that could be used in a spinal construct as shown in FIG. 1.

FIG. 3 is a side cross-sectional view of a transverse connector with set screws partially threaded therein according to one embodiment of the disclosure.

FIG. 4 is a side cross-sectional view of a portion of the transverse connector and set screw illustrated in FIG. 3.

FIG. 5 is a side view of the set screw illustrated in FIGS. 3 and 4.

FIG. 6 is a cross-sectional view of the set screw taken along line VI-VI of FIG. 5.

FIG. 7 is a side cross-sectional view of a transverse connector with the set screws partially threaded therein according to another embodiment of the disclosure.

FIG. 8A is a side view of the set screw illustrated in FIG. 7.

FIG. 8B is a perspective view of the set screw illustrated in FIG. 7.

FIG. 9 is a side cross-sectional view of a portion of the transverse connector and set screw illustrated in FIG. 7.

FIG. 10 is a perspective view of a transverse connector with set screws partially threaded therein according to another embodiment of the disclosure.

FIG. 11 is a side cross-sectional view of the transverse connector and set screws illustrated in FIG. 10.

FIG. 12 is a side cross-sectional view of a portion of the transverse connector and set screw as illustrated in FIG. 11.

FIG. 13 is a exploded perspective view of a portion of a transverse connector and set screw according to yet another embodiment of the disclosure.

FIG. 14 is a side cross-sectional view of a portion of a transverse connector and set screw as illustrated in FIG. 13.

FIG. 15 is a side perspective view of a spinal connector used to connect an elongated rod to a threaded pedicle bolt/screw.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

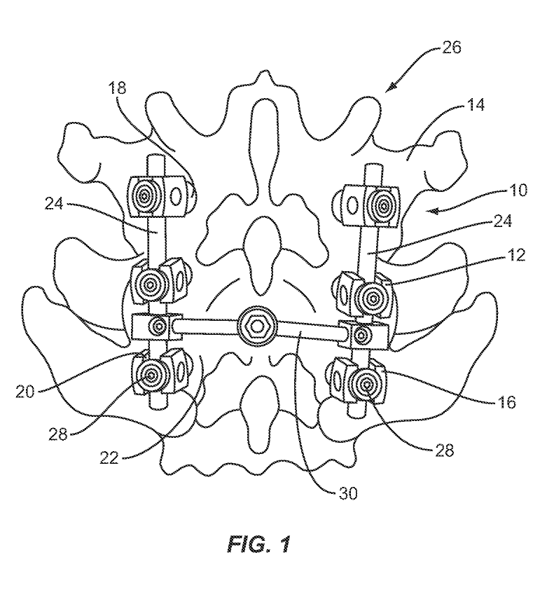

Referring to FIG. 1, a construct or spinal stabilization system 10 is illustrated that is utilized to treat a variety of spinal conditions. In this form, construct 10 includes a plurality of multi-axial screws 12 that are inserted into select portions of vertebrae 14 to be instrumented. In this representative form shown, the multi-axial screws 12 include a screw head 16 and a threaded shaft 18 extending downwardly from the screw head 16. As illustrated, screw head 16 comprises a U-shaped member or cradle that includes two opposing internally threaded sidewalls 20. In one form, multi-axial screws 12 are preferentially made from titanium and include top-loading screw heads. In this form, the threaded shaft 18 of the multi-axial screws 12 is inserted through a pedicle 22 of the vertebrae 14 and into a portion of the vertebrae 14.

As shown in FIG. 1, after insertion of the multi-axial screws 12 in the desired locations, a rod 24 is inserted into the U-shaped screw heads 16 such that the rod 24 extends along a sagittal plane of the spine 26. In this example, rod 24 is positioned in three U-shaped screw heads 16 but other numbers of multi-axial screws 12 may be used in other surgical procedures. The rods 24 preferentially run substantially parallel to one another along the sagittal plane of the spine 26. Once the rods 24 are in proper position, set screws or plugs 28 are screwed into the screw heads 16 thereby securing the rods 24 to the multi-axial screws 12. To provide further stabilization of the spine 26, a transverse connector 30 is connected to each respective rod 24 that runs substantially perpendicular to the rods 24. As such, in this form the transverse connector 30 runs along an axial plane of the spine 26. Although only one transverse connector 30 is illustrated in FIG. 1, one or more transverse connectors 30 may be used in other surgical procedures. As with the multi-axial screws 12, the rods 24 and transverse connectors 30 may be manufactured from titanium or any other biocompatible material that is strong enough to provide the stabilization desired to be obtained from the surgical procedure. Other spinal constructs utilize threaded screws having smooth upper shafts or posts and in this case a spinal connector would be used to connect the spinal rods to the threaded post screws. Such a spinal connector will be discussed in greater detail below.

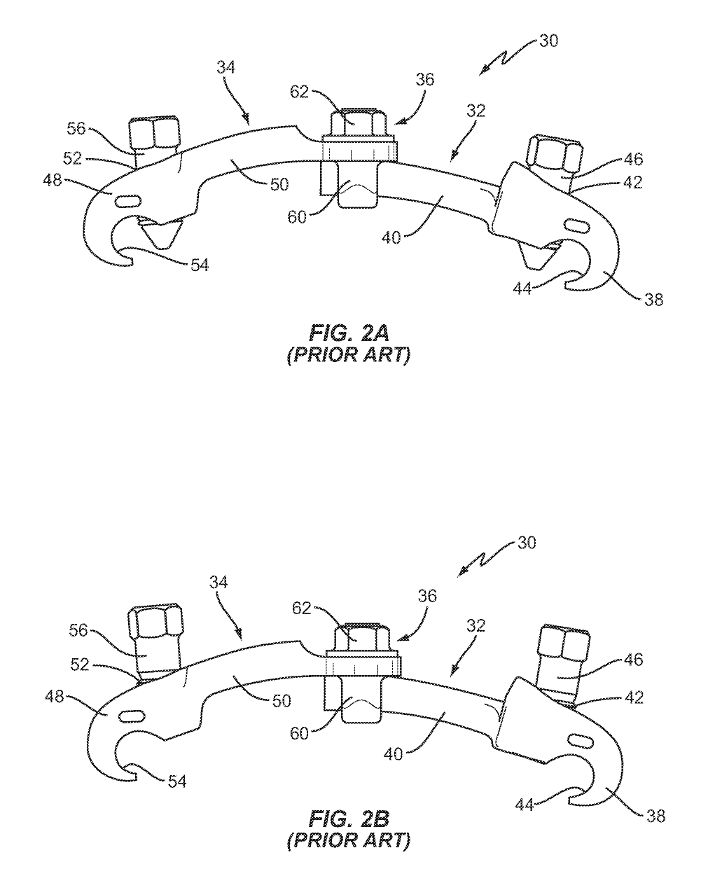

Referring to FIGS. 2A and 2B, a prior art transverse connector 30 is shown. The transverse connector 30 is shown having a first arm 32, a second arm 34, and an eyebolt 36. The first arm 32 includes a hook segment 38 and a rod or extension segment 40. The hook segment 38 includes a threaded aperture 42 that extends vertically or downwardly through the hook segment 38 and into a rod channel 44 of the hook segment 38. In this form, rod channel 44 comprises a passageway through the hook segment 38. Referring collectively to FIGS. 1, 2A and 2B, the rod channel 44 is configured and sized to receive one of the rods 24. Once the rod 24 is positioned in the hook 48, a set screw 46 is positioned or screwed into the threaded aperture 42 and tightened thereby securing the first arm 32 to the rod 24. The rod segment 40 has a generally circular cross-sectional configuration in this representative example, but may have other cross-sectional configurations (e.g.—rectangular, square, octagonal, and so forth) in other representative forms.

The second arm 34 includes a hook segment 48 and an arm or extension segment 50. As with the first arm 32, the hook segment 48 includes a threaded aperture 52 that extends vertically or downwardly through the hook segment 48 and into a rod channel 54 of the hook segment 48. In this form, rod channel 54 comprises a passageway through an end of the hook segment 48. Referring collectively to FIGS. 1, 2A and 2B, the rod channel 54 is configured and sized to receive one of the rods 24. Once the rod 24 is positioned in the rod channel 54, a setscrew 56 is positioned or screwed into the threaded aperture 52 and tightened thereby securing the second arm 34 to the rod 24. A distal end 58 of the arm segment 50 includes an eye 60 that is used to secure the second arm 34 to the eyebolt 36. In particular, a nut 62 is used to secure the second arm 34 to the eyebolt 36. In this representative form, the arm segment 50 has a generally circular cross-sectional configuration, but may have other cross-sectional configurations (e.g.—rectangular, square, octagonal, oval and so forth) in other representative forms.

As shown in FIG. 2A, the transverse connector 30 is shown with the set screws 46 and 56 completely threaded into the threaded apertures 42 and 52 so that the rod channels 44 and 54 are blocked such that a rod 24 can not be inserted within the rod channels 44 and 54. This is typically how a transverse connector of this type would be shipped from the manufacturer as discussed above. Before the surgeon can use the transverse connector as shown in FIG. 2A, the surgeon and/or hospital staff while have to loosen the sets screws 46 and 56 as shown in FIG. 2B. Referring to FIG. 2B, the transverse connector 30 is shown with the set screws 46 and 56 loosened such that the rod channels 44 and 54 are not blocked. However, as discussed above, if the transverse connector 30 is shipped in this fashion there are serious concerns that the set screws 46 and 56 could accidentally fall out of the transverse connector 30 during shipment and/or with later handling of the product at the customer's place of business. The surgeon and/or the hospital staff will then have to relocate the set screws for insertion back into the transverse connector before it would be ready to use in the surgical operation. Furthermore, when re-inserting the set screws into threaded apertures 42 and 52 it is possible to cross-thread the set screws thereby making the transverse connector unusable. This whole process of preparing the transverse connectors for use in the operating room causes surgeon frustration and possibly damaged implants and additional cost to the customers and manufacturers.

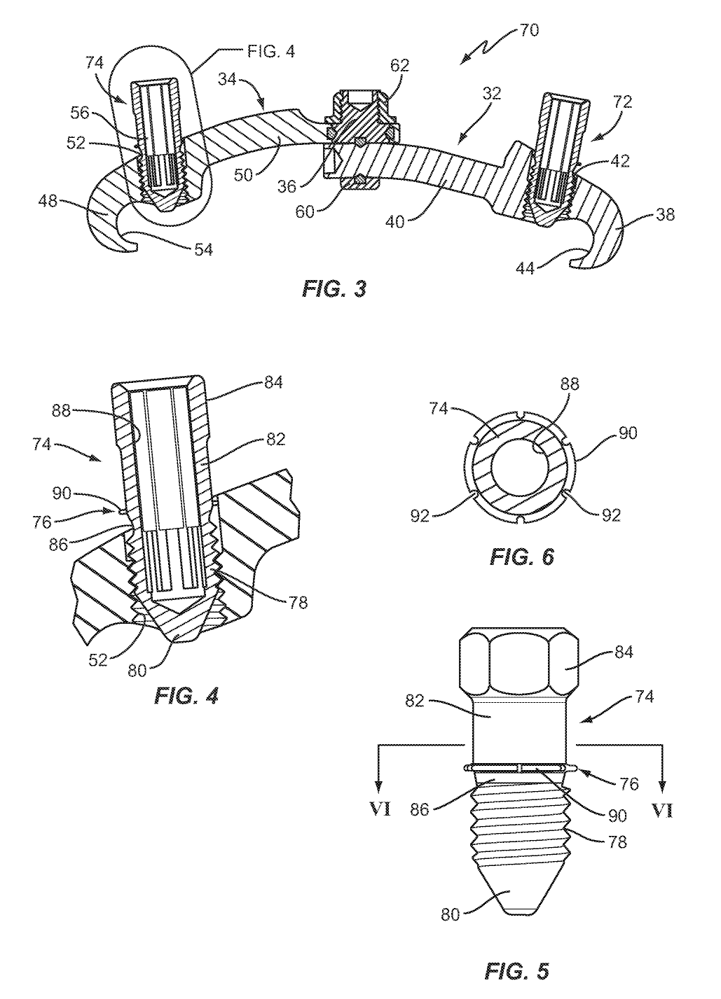

Referring to FIGS. 3 thru 6, the transverse connector 70 of the present invention is shown in greater detail. The transverse connector 70 is similar to the prior art transverse connector 30 and where like parts are the same the same reference numerals are used. However, in this representative form, the transverse connector 70 is provided with set screws 72 and 74 that include a friction member 76 that allows the set screws 72 and 74 to be partially threaded into and then held in such a position within the threaded apertures 42 and 52 until the surgeon is ready to install the transverse connector 70 onto the spinal rods 24 in a spinal surgical operation. The level of resistance to movement provided by the friction member 76 is selected to be of sufficient strength for prevention of any further easy rotation and/or loosening of the set screw during transportation, shipping or any pre-implantation manipulation by the surgeon any/or hospital staff, but to still allow a surgeon to tighten the set screws when using the transverse connector 70 in a surgical operation. For ease of discussion, the disclosure will be further described with respect to a single set screw 74 as shown in FIGS. 3 and 4. The set screw 72 will work in the same fashion as that described below with respect to set screw 74.

As shown in FIGS. 4 and 5, the set screw 74 is a break-off head type set screw. These screws are well known in the art typically having a threaded portion 78 with a depending rod engaging end 80 and a break-off head portion 82 having a driving portion 84 configured to receive a screw driver or the like for rotating the set screws into contact with the spinal rod 24. The manner in which break-off set screws work is that a surgeon would tighten the set screw until the rod engaging end 80 contacts the rod 24 and with continued rotation by the surgeon, a sufficient rotational force would be generated to separate the break-off head portion 82 at the break point 86 from the threaded portion 78 and rod engaging end 80 which would remain in the transverse connector 70 securely holding the rod 24 within the rod channel 54 thereof. An internal socket portion 88 is provided through the driving portion 84 and into the threaded portion 78 to allow a socket driver (not shown) to be used to later remove the set screw 74 from the transverse connector 70 if it becomes necessary even after the break-off head portion 82 has been separated from the set screw 74.

Referring specifically to FIGS. 4 and 5, the friction member 76 is shown in greater detail. The friction member 76 may be a deformable skirt 90 provided about the circumference of the set screw 74. The deformable skirt 90 will be elastically deformed when initially threading the set screw 74 into the threaded aperture 52 by the manufacturer such that the skirt 90 will then hold the set screw 74 in such a position until use by the surgeon. In this form, the skirt 90 is shown to be integral with the body of the set screw 74. However, it may also be desirable to provide a groove about the circumference of the set screw and to insert a separate deformable skirt member into such groove. Furthermore, as shown herein the deformable skirt 90 is provided about the break-off head portion 82 at a point above the break point 86 of the break-off set screw 74. When the break-off head portion 82 is separated from the threaded portion 78 the deformable skirt 90 will be removed from the surgical site by the surgeon to prevent any contaminating particles of the friction member 76 being left in the surgical site. It may be desirable to provide the deformable skirt 90 about the set screw at a point below the break point 86 so the skirt will remain with the threaded portion 78 after the break-off head portion 82 is removed from the set screw 74.

Referring to FIG. 6, it may be desirable to provide the deformable skirt 90 with a plurality of segmentations 92 to provide for a greater elastic deformation of the skirt 90 and to reduce the required torque to overcome the plastic deformation of the skirt to allow the set screw 74 to be more easily initially installed within the transverse connector 70 by the manufacturer. The skirt 90 with segmentations 92 would then also be easier to install by the surgeon when continued rotation of the set screw is necessary to fully tighten the set screw 74 into the transverse connector 70 to connect the connector 70 to a spinal rod 24 in a spinal operation.

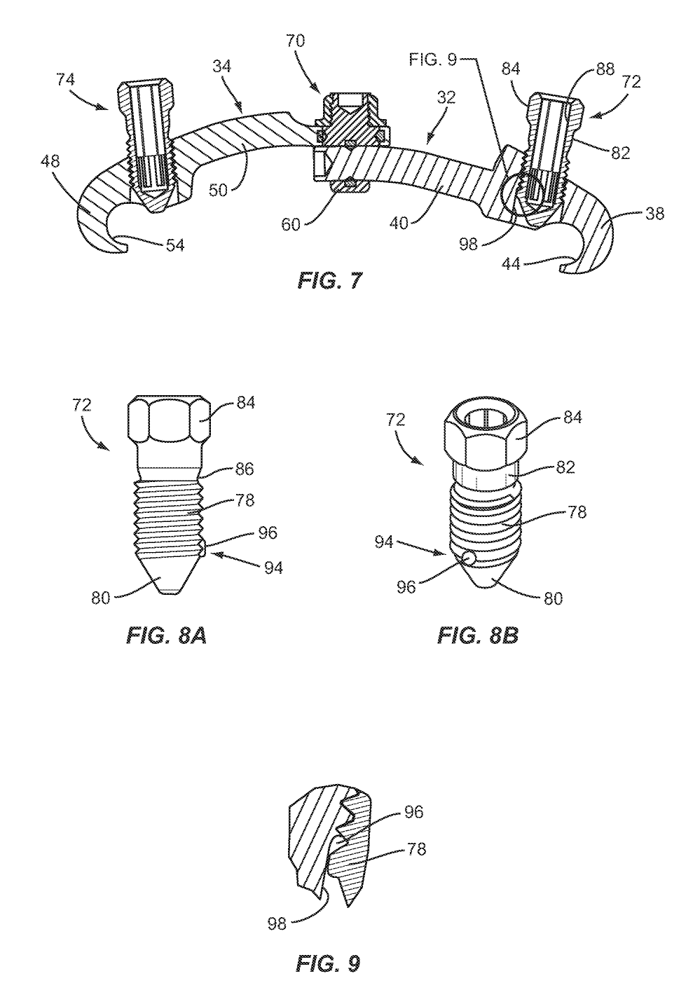

Referring to FIGS. 7, 8A, 8B and 9, another form of the present invention is shown. The transverse connector 70 is shown in substantially the form as discussed above except that set screw 72 is now provided with another embodiment of the friction member 94. In this form, friction member 94 is a compressible patch 96 of material placed within the threaded portion 42 near the rod engaging end 80 of set screw 72. The patch may be made of UHMWPE, silicone, or other implant grade compressible material. When the set screw 72 is initially threaded into the threaded aperture 42 the compressible patch 96 will be compressed between the threaded aperture 42 of the transverse connector 70 and the threaded portion 78 of the set screw 72 as shown with specific reference to FIG. 9. This compression will hold the set screw 72 within the threaded aperture 42 against any further rotation to ensure that the set screw stays within the transverse connector 70 during transportation and any pre-implantation manipulation by the surgeon and/or hospital staff. When a surgeon is ready to install the transverse connector 72 onto a pair of spinal rods 24, an appropriate driver will be used to overcome the compressive forces provided by the compressible patch 96 to fully install the set screw 72 into the transverse connector 70 to securely grip the spinal rod 24. A counterbore 98 is provided immediately below the threaded aperture 42 of the transverse connector 70 so as to prevent any further friction by the patch 96 with the set screw rod engaging end 80 is contacting the spinal rod 24.

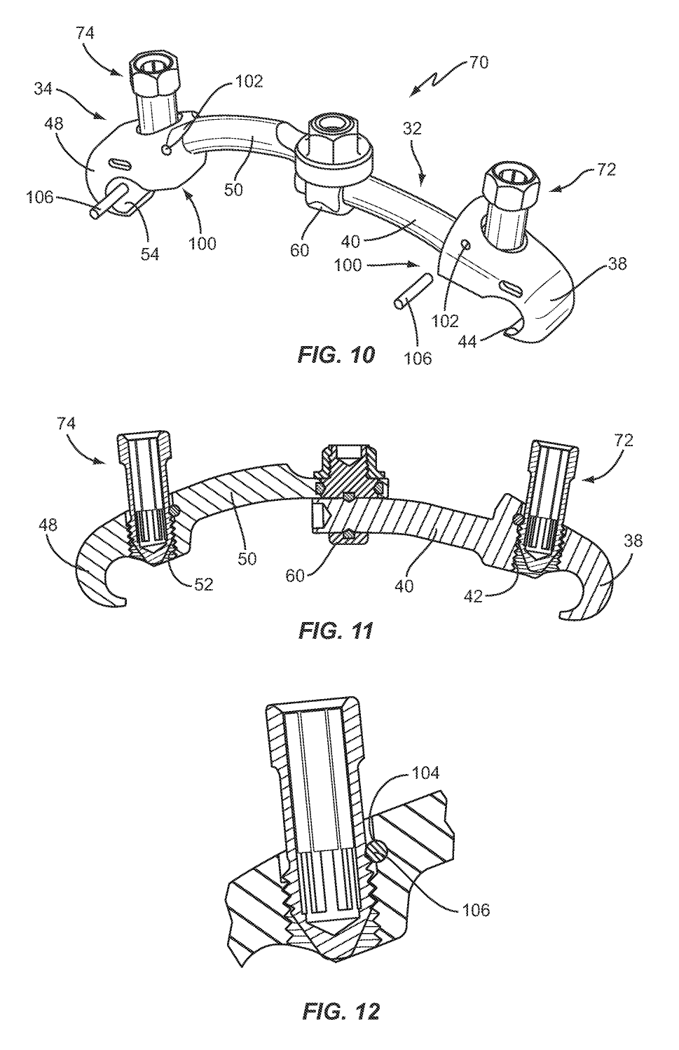

Referring to FIGS. 10 thru 12, another form of the present invention is shown. The transverse connector 70 is shown in substantially the form as discussed above except that the transverse connector 70 is provided with another embodiment of the friction member. Friction member 100 will be described in relation to the second arm 34 having a hook segment 48 and an arm or extension segment 50. The hook segment 48 includes a threaded aperture 52 that extends vertically or downwardly through the hook segment 48 and into a rod channel 54 of the hook segment 48. In this form, rod channel 54 comprises a passageway through an end of the hook segment 48 which is configured and sized to receive one of the rods 24. The friction member 100 for use with the first arm 32 would minor that discussion here with respect to the second arm 34. Referring to FIG. 10, the hook segment 48 is provided with a bore 102 which intersects the threaded aperture 52 of the second arm 34 as shown at 104. A retaining pin 106 is provided for insertion in bore 102 to retain the set screw 74 within the threaded aperture 52. The retaining pin can be manufactured out of any medical grade metal or polymeric material which is softer than the threaded portion 78 of set screw 74. The transverse connector 70 would be assembled by first threading the set screw 74 into the transverse connector until the threaded portion 78 thereof is past the intersecting bore 102. The compressible pin 106 would then be inserted into the bore 102. The set screw 74 would then be unscrewed from the transverse connector 70 until the threaded portion 78 contacts and compresses the pin 106 as specifically shown in FIG. 12. The compression between the pin 106 and set screw threaded portion 78 would act to hold the set screw 74 in place within the threaded aperture 52 during transportation and any pre-implantation manipulation of the transverse connector 70. The pin 106 may be held in place within the bore 102 by friction or by heat staking either end of the pin 106 where it exits bore 102 on the second arm 34.

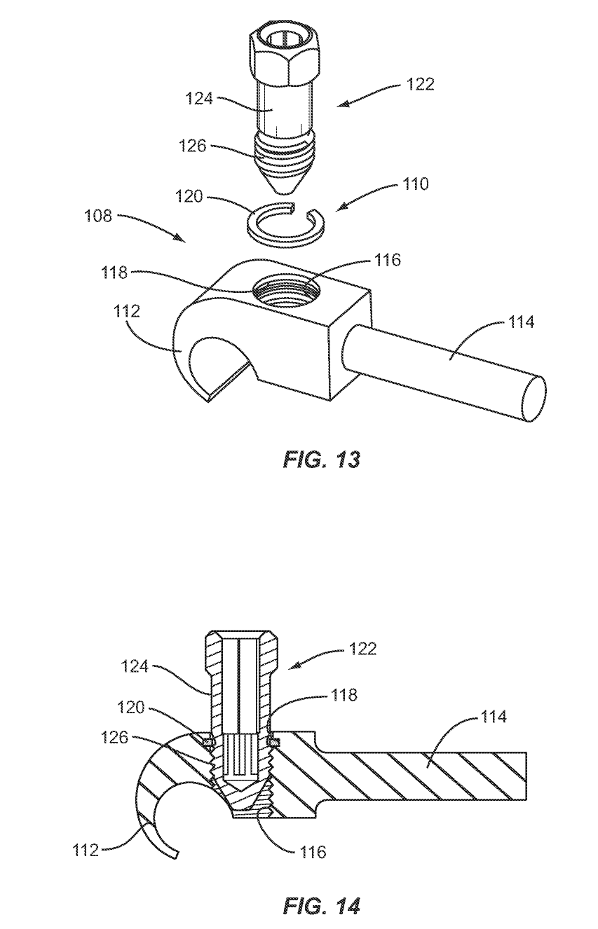

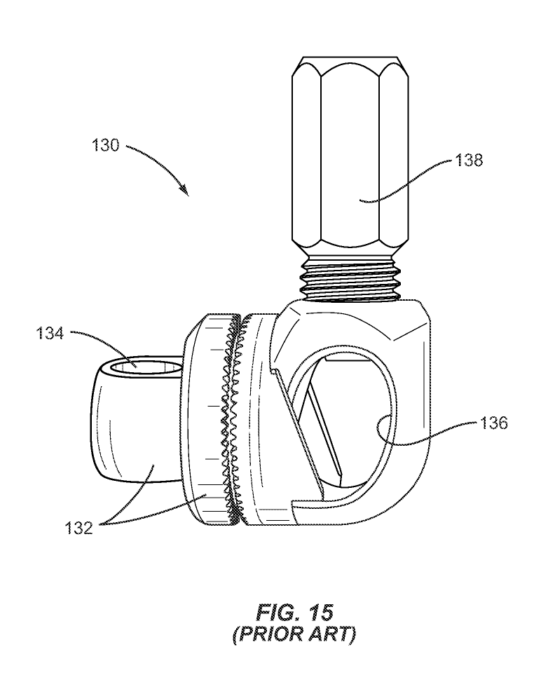

Referring to FIGS. 13 and 14, yet another form of the present invention is shown. The transverse connector is shown with respect to a single arm 108 for connection to a spinal rod 24. A second arm would be provided attached to the single arm 108 to make a fully functioning transverse connector. Friction member 110 is described in relation to the single arm 108 having a hook segment 112 and an arm or extension segment 114. The hook segment 112 includes a threaded aperture 116 that extends vertically or downwardly through the hook segment 112 and into a rod channel of the hook segment 112. As discussed above, rod channel 54 comprises a passageway through an end of the hook segment 112 which is configured and sized to receive one of the rods 24. Friction member 110 includes a groove 118 provided in the threaded aperture 116 adjacent the entrance or top of the threaded aperture 116. A snap ring 120 is provided in groove 118. The set screw 122 is provided with a threaded portion 126 and a drive portion 124. When the set screw 122 is threaded into the threaded aperture 116, the snap ring 120 is compressed against the threaded portion 126 of the set screw 122. The set screw 122 would be threaded into the threaded aperture 116 by the manufacturer until just the last of the threaded portion 126 of the set screw 122 contacts the snap ring 120 as shown in FIG. 14. The surgeon would then need to only apply further rotation torque on the set screw with an appropriate driver to fully install the transverse connector unto a spinal rod. Alternatively, the manufacturer could first install the set screw 122 into the threaded aperture 116 until the threaded portion 116 is past the groove 118. The snap ring 120 could then be installed in the groove 118 and the set screw then be unthreaded from the transverse connector until the threaded portion 126 of the set screw 122 contacts and is compressed against the snap ring 120. The surgeon would then install the transverse connector onto a spinal rod in the same fashion as discussed above.

Referring to FIG. 15, yet another form of the present invention is shown. As discussed above when utilizing a spinal construct having threaded post pedicle screws (not shown) instead of the multi-axial pedicle screws discussed above, a spinal connector 130 would be used to connect the rod 24 to the threaded post screws. Spinal connector 130 is simply one type of connector that may be used in such a situation. In this example, the connector has an eyebolt arrangement 132 that has a first bore 134 for receiving the smooth post of the screw therethrough. The connector 130 further has a second bore 136 for receiving the rod 24 therethrough and a set screw 138. The set screw 138 may be of the break-off head variety or not. In either case, when the set screw 138 is threaded against a rod (not shown in FIG. 15) the rod would push against the eyebolt arrangement 132 to cause the post of the threaded post screw to be captured within the first bore 134. The set screw 138 would also affix the rod within the second bore 136 of the connector 130. The various embodiments of the present invention as discussed with reference to FIGS. 4 thru 14 above would all work equally as well with respect to the set screw 138 of spinal connector 130 shown in FIG. 15. It would be desirable for the manufacturer to ship the spinal connector part with the set screw partially threaded into the connector without worry that the set screw would accidentally back out of the connector. All of the embodiments of the disclosed invention could be utilized with the set screw 138 of the spinal connector 130 to provisionally hold the set screw in place until a surgeon is ready to utilize the connector in a spinal operation.

The various embodiments that have been described above as utilizing break-off type set screws. However, the retention mechanism for rod locking set screws as described herein may also utilize non break-off set screws. It is equally important that such set screws not become loose within the transverse connector during shipment or any pre-implantation manipulation by the surgeon and/or the hospital staff. All of the various embodiments of the friction members discussed herein would work equally as well on both transverse connectors utilizing break-off and non break-off type set screws.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “top” and “bottom” refer to the direction in reference to the position of the device as shown in the Figures.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A connector having a set screw for connecting a spinal rod to the connector, comprising:
a body having a rod receiving portion and a threaded aperture intersecting the rod receiving portion;
a set screw having a threaded portion partially received in said threaded apertures of said body; and
means for inhibiting rotational movement of said partially inserted set screw within said threaded aperture of said body to prevent accidental removal of the set screw from the body during shipment or pre-implantation manipulation of the connector prior to connection to the spinal rod.

2. A connector of claim 1, wherein said means for inhibiting comprises a set screw having a friction member provided about its circumference providing a friction fit within the threaded aperture, the friction member providing initial resistance to continued rotation of the partially inserted set screw, such initial resistance being overcome by use of additional rotational force on said set screw.

3. A connector of claim 2, wherein said set screw comprises a threaded portion with a rod engaging nose and a break-off head portion with a drive portion configured to receive an instrument for rotating the set screw, the friction member being provided about the break-off head portion such that when the break-off head portion is separated from the threaded portion the friction member is removed from the transverse connector.

4. A connector of claim 2, wherein the friction member is a deformable skirt provided about the circumference of the set screw, the deformable skirt being elastically deformed upon initial threading of the set screw within the threaded aperture to initially hold the set screw within the aperture.

5. A connector of claim 4, wherein the set screw is provided with a groove about its circumference, a separate deformable skirt being contained in said groove.

6. A connector of claim 4, wherein the deformable skirt is provided with a plurality of segmentations about its circumference to provide for increased elastic deformation of said skirt to ease both initial installation of said set screw within said threaded aperture and continued installation of said set screw to capture the rod within said connector.

7. A connector of claim 1, wherein the means for inhibiting comprises a compressible patch of material provided within the threaded portion of said set screw such that upon initial installation of said set screw within the threaded aperture of the said body, the compressible patch is compressed between the set screw and the threaded aperture, and upon continued rotation of said set screw the compressible patch will vacate the threaded aperture to provide easy final installation of said set screw to capture the rod within said connector.

8. A connector of claim 1, wherein the means for inhibiting comprises a bore transverse to said threaded aperture of said body, said bore intersecting the threaded aperture and having a compressible retaining pin provided in said bore, the retaining pin providing initial resistance against continued rotation of said set screw, however upon continued rotation of said set screw the threaded portion of said set screw will pass said retaining pin to allow for easy installation of said set screw to capture the rod within said connector.

9. A connector of claim 1, wherein the means for inhibiting comprises a snap ring being provided within a circumference groove provided within the threaded aperture of said body, the snap ring being compressible to inhibit rotational movement of said partially inserted set screw within said threaded aperture of said body to prevent accidental removal of the set screw from the body during shipment or pre-implantation manipulation the connector prior to connection to the spinal rod.

10. A transverse connector for connecting a pair of spinal rods together, comprising:
a first arm having a rod receiving portion and a threaded aperture intersecting the rod receiving portion;
a second arm connected to the first arm and having a rod receiving portion and a threaded aperture intersecting the rod receiving portion;
a set screw partially received in each of said respective threaded apertures of said first arm and second arm; and
means provided for inhibiting rotational movement of said partially inserted set screw within said threaded apertures of the first arm and second to prevent the removal of the set screws from the first and second arms during shipment or pre-implantation manipulation the transverse connector prior to connection to the spinal rods.

11. A transverse connector of claim 10, wherein said means for inhibiting comprises the set screw being a friction member provided about its circumference providing a friction fit within the threaded aperture, the friction member providing initial resistance to continued rotation of the partially inserted set screw, such initial resistance being overcome by use of additional rotational force on said set screw.

12. A transverse connector of claim 11, wherein said set screw comprises a threaded portion with a rod engaging nose and a break-off head portion with a drive portion configured to receive an instrument for rotating the set screw, the friction member being provided about the break-off head portion such that when the break-off head portion is separated from the threaded portion the friction member is removed from the transverse connector.

13. A transverse connector of claim 11, wherein the friction member is a deformable skirt provided about the circumference of the set screw, the deformable skirt being elastically deformed upon initial threading of the set screw within the threaded aperture to initially hold the set screw within the aperture.

14. A transverse connector of claim 13, wherein the set screw is provided with a groove about its circumference, a separate deformable skirt being contained in said groove.

15. A transverse connector of claim 13, wherein the deformable skirt is provided with a plurality of segmentations about its circumference to provide for increased elastic deformation of said skirt to ease both initial installation of said set screw within said threaded aperture and continued installation of said set screw to capture the rod within said transverse connector.

16. A transverse connector of claim 10, wherein the means for inhibiting comprises a compressible patch of material provided within the threaded portion of said set screw such that upon initial installation of said set screw within the threaded aperture of the said body, the compressible patch is compressed between the set screw and the threaded aperture, and upon continued rotation of said set screw the compressible patch will vacate the threaded aperture to provide easy final installation of said set screw to capture the rod within said transverse connector.

17. A transverse connector of claim 10, wherein the means for inhibiting comprises a bore transverse to said threaded aperture of said body, said bore intersecting the threaded aperture and having a compressible retaining pin provided in said bore, the retaining pin providing initial resistance against continued rotation of said set screw, however upon continued rotation of said set screw the threaded portion of said set screw will pass said retaining pin to allow for easy installation of said set screw to capture the rod within said transverse connector.

18. A transverse connector of claim 10, wherein the means for inhibiting comprises a snap ring being provided within a circumference groove provided within the threaded aperture of said body, the snap ring being compressible to inhibit rotational movement of said partially inserted set screw within said threaded aperture of said body to prevent accidental removal of the set screw from the body during shipment or pre-implantation manipulation of the transverse connector prior to connection to the spinal rod.

19. A connector having a set screw for connecting a spinal rod to the connector, comprising:
a body having a rod receiving portion and a threaded aperture intersecting the rod receiving portion;
a set screw having a threaded portion partially received in said threaded apertures of said body; and
means provided for inhibiting rotational movement of said partially inserted set screw within said threaded aperture of said body to prevent accidental removal of the set screw from the body during shipment or pre-implantation manipulation of the connector prior to connection to the spinal rod, said means for inhibiting rotational movement being overcome by use of additional rotational force on said set screw to capture the rod within said connector.

20. A connector of claim 19, wherein the connector is a transverse connector for connecting a pair of spinal rods together, the transverse connector having first and second arms each having a rod receiving portion and a threaded aperture intersecting the rod receiving portion, a set screw partially received in each of said respective threaded apertures of said first and second arms.
